(19) United States
(12) Patent Application Publication
(10) Pub. No.: US 2011/0177126 A1
(43) Pub. Date: Jul. 21, 2011

(54) BACTERIA AND DERIVED PRODUCTS TO REINFORCE THE BODY DEFENCES AND TO REDUCE THE RISK OF DISEASE

(75) Inventors: Yolanda Sanz Herranz, Valencia (ES); Giada De Palma, Valencia (ES); Esther Sánchez Sánchez, Valencia (ES); Marcela Susana Medina, Valencia (ES)

(73) Assignee: CONSEJO SUPERIOR DE INVESTIGACIONES CIENTIFICAS, Madrid (ES)

(21) Appl. No.: 13/054,652

(22) PCT Filed: Jul. 14, 2009
(86) PCT No.: PCT/ES2009/070285

§ 371 Date: Apr. 11, 2011

(30) Foreign Application Priority Data
Jul. 15, 2008 (ES) P200802102

(51) Int. Cl. A61K 39/02; C12N 1/20; A61K 35/74; C07G 99/00; A61P 31/00; A61P 3/00; A61P 37/02; A23L 1/28; A23L 1/48

(52) U.S. Cl. 424/234.1; 435/252.1; 424/93.4; 260/1; 426/61; 426/655

(57) ABSTRACT

This invention relates to a new strain of the genus Bifidobacterium and the components of its supernatants of cultivation in the form of various preparations (functional and new foods, probiotics, symbiotics, supplements, nutraceutics and medications) with the purpose of improving the defences and reducing the risk of disease. Its mechanisms of action include: (i) regulation of the intestinal glycosylation favouring that of healthy individuals, (ii) modulation of the interactions between the epithelial cells and the intestinal microbiotics, favouring the adhesion of beneficial bifidobacteria and (iii) regulation of the immunological responses. The products included are stable in gastrointestinal and technological conditions which guarantee their exploitation.

The present invention belongs to the field of the food and pharmaceutical industry. In particular, this invention is related to the probiotics and derived products in the form of functional foods and new foods, probiotics, symbiotics, nutraceotics or nutritional supplements and pharmaceutical formulations with clinical applications.

### STATE OF THE ART

The mucous membrane of the gastrointestinal tract is one of the main zones of contact with potentially harmful environmental agents (bacteria, viruses, toxins and allergens). As a result of that, the gastrointestinal tract has developed complex mechanisms for defence by applying an important additional protective function of the digestive system. One starts to acquire microbiotics since birth and its composition has a great repercussion on the development of the physiological functions of the individual as well as on the predisposition to suffering infections y pathologies on an immunological basis. The genus Bifidobacterium is one of the predominant ones in the colon and represents normally between 3% and 7% of the intestinal microbiotics in adults and up to 91% in newborns (Harmsen et al., 2000. Analysis of intestinal flora development in breast-fed and formula-fed infants by using molecular identification and detection methods. J Pediatr Gastroenterol Nutr. 2000 January; 30(1): 61-7.). The low susceptibility of breastfed children to diseases is considered related to the predominance of intestinal bifidobacteria. These bacteria contribute to the state of health of the individual by means of various mechanisms such as the exclusion of pathogens and the regulation of the immunological responses and the metabolic functions of the body (Boirivant and Strober 2007. The mechanism of action of probiotics. Curr Opin Gastroenterol. 23:679-692; Sanz et al. 2007 Probiotics as drugs against human gastrointestinal infections. Recent patents Anti-Infect Drug Disc. 2; 2: 148-56; Toba Takahiro et al. 2007. Bifidobacterium or lactic acid bacterium having effect of preventing infection via ss-defensin and food/pharmaceutical composition containing the same. WO2007023912; Petay et al., 2006. Immunomodulatory product obtained from a Bifidobacterium culture and compositions containing the same. EP1615657, etc.). As a consequence, the strains of the species Bifidobacterium constitute one of the main probiotics marketed for human consumption for different purposes.

The mucus covering the intestinal mucous membrane, the epithelial cells, the bloodstream that irrigates it and the secretions (phospholipids, bile, antimicrobial peptides, etc.) constitute jointly an innate protective barrier of a physicochemical nature (Bourlioux et al. 2002. The intestine and its microflora are partners for the protection of the host. Am J Clin Nutr 2003; 78, 675-683). The intestinal mucus, integrated by the mucins, represents one of the first mechanisms of defence prior to the activation of the immunocompetent cells. The mucins are glycoproteins with high molecular weight secreted by the epithelial cells (Singh and Hollingsworth 2006. Cell surface-associated mucins in signal transduction Trends in Cell Biol. 16, 467-476). The mucins can consist of the following carbohydrates: N-acetylglucosamine, galactose, N-acetylgalactosamine, fucose, N-acetylneuraminic acid, sialic acid, mannose, glucose and xylose (Freitas et al. 2005. Indigenous microbes and their soluble factors differentially modulate intestinal glycosylation steps in vivo. Histochem. Cell. Biol. 124: 423-433). The mucins are involved in the cell recognition processes, the adhesion and invasion of pathogens, the transmission of signals and in the proliferation and differentiation of the tumour cells (Sudha et al. 2001. Adherence of Shigella dysenteriae 1 to human colonic mucin Curr. Microbiol. 42: 381-387; Leteurtre et al. 2004. Differential mucin expression in colon carcinoma HT-29 clones with variable resistance to 5-fluorouracil and methotrexate Biol. of the cell 96: 145-151; Singh and Hollingsworth 2006. Cell surface-associated mucins in signal transduction Trends in Cell Biol. 16: 467-476). The expression and the composition of the mucins are regulated genetically in every individual; in addition to that, it has been described how the intestinal microbiotics can contribute to their regulation by modifying the gene expression of the enzymes of the host involved in their synthesis (for example, glycosiltransferases and glycosidases) or also by their utilisation as nutrients (Hooper and Gordon. 2001. Commensal host-bacterial relationships in the gut. Science; 292: 1115-1118.). By using germ-free animals and conventional animals, it has been demonstrated that the process of colonisation by commensal bacteria is favourable to the synthesis of glycoconjugated fucosylates [fucose α(1,2)-galactose β] in the small intestine (Hooper and Gordon. 2001. Commensal host-bacterial relationships in the gut. Science; 292: 1115-1118). Later on, it was demonstrated that Bacteroides thetaiotaomicron VPI-5482 secreted a soluble compound that modified the process of galactosylation in cell cultures and animal models; however, its possible beneficial effects on health have not been investigated (Freitas et al., 2001; Freitas et al., 2005. Indigenous microbes and their soluble factors differentially modulate intestinal glycosylation steps in vivo. Histochem. Cell. Biol. 124: 423-433). The changes in the glycosylation can favour the adhesion and retention of bacteria and antigens which otherwise would pass through the intestinal lumen without interacting with the epithelium. A certain pattern of the glycosylation favours the adhesion and colonization of commensal bacteria with beneficial effects on health, while another one can increase the adhesion of pathogens and the susceptibility to microbial infections or favour inflammation diseases (allergies, intestinal inflammation diseases, autoimmune diseases, cancer, etc.). For example, the receptors, which permit the adhesion of Escherichia coli, contain galactose and sialic acid and those permitting the adhesion of Helicobacter pylori contain fucose due to which the patterns of glycosylation rich in these residues would be good receptors for these pathogens favouring their colonisation. On the other hand, the increase of the sialic acid content in the terminal ends and the reduction of the sulfatation are associated with intestinal inflammatory diseases and cancer (Campbell et al., 2001. Altered glycosylation in inflammatory bowel disease: a possible role in cancer development. Glycoconj J. 18, 851-8). In some studies, the changes in the intestinal glycosylation have been detected by the use of lectins which are proteins associating specifically certain residues of carbohydrates and acting as markers of the composition of the glycoconjugates. In the jejunum of individuals with a sensitivity to gluten, association of the lectin UEA (L-fucose) has been detected, but not in healthy people, which indicates that the corresponding residues of carbohydrates are characteristic of the mucus of the studied patients (Vecchi et al., 1989. Evidence of altered structural and secretory glycoconjugates in the jejunal mucosa of patients with gluten sensitive enteropathy and subtotal villous atrophy. Gut. 30, 804-10). More recent studies have demonstrated that the mucus of celiac patients shows a particular structure leading to a strong tinting with lectin UEA (α 1,2-fucose) and not with the PNA (Gal β [1→3] GalNAc) which is characteristic for healthy people (Forsberg et al. 2004. Presence of bacteria and innate immunity of intestinal epithelium in childhood celiac disease. Am. J. Gastroenterol. 99:894-904).

Many beneficial effects have been attributed to strains of the genus Bifidobacterium; however, the function of different species of this genus in the glycosylation of the intestinal mucus as a mechanism for modification of the process of colonisation of the beneficial microbiotics and its influence on the risk for disease has not been described as it is proposed in the present invention. The bioactive compounds of bacterial origin involved in this process, produced by strains of the genus Bifidobacterium, which could constitute a basis for applications for improving the state of the health, have also not been characterised. The studies conducted until now were concentrated only on characterisation of the enzymes involved in the degradation of the mucus as a source of carbon for the growth of these bacteria in the colon (Ruas-Madiedo et al. 2008. Mucin degradation by Bifidobacterium strains isolated from the human intestinal microbiota. Appl Environ Microbiol. Jan 25, 2008) or in the utilisation of the prebiotics which, administered through the diet, are favourable to their growth and their possibilities for survival (Viscomi, et al. 2006. SI1398369T; Vriesema et al. 2006. WO2006112714). On the other hand, there is a method for selection of microorganisms which modulate the glycosylation, including lactic bacteria; however, neither the compounds responsible for these processes have been characterised nor the beneficial effects these modifications can have on improving the health have been demonstrated (Jean-Michel et al., 2004. Micro-organisms with glycosylation modulating action of intestinal cell surface. 20040058409).

The intestinal microbiotics also regulate specific aspects of the innate and acquired immunity, protecting the host from infections and processes of chronic inflammation (for example, Crohn's disease and celiac disease). The epithelial cells and the cells presenters of antigens of the innate immune system have cellular receptors (‘toll-like receptors’ and ‘NOD-like receptors’) capable of discriminating between the commensal and pathogenic microbiotics, inducing the synthesis of certain mediators of the innate immune response (cytokines, chemokines, molecules of adhesion, etc.) and of suitable adaptive responses with the purpose of fighting against the pathogen (Werner and Haller. 2007. Intestinal epithelial cell signalling and chronic inflammation: From the proteome to specific molecular mechanisms. Mutat Res. 1; 622(1-2):42-57). The moderate induction of the synthesis of proinflammatory chemokines and cytokines for beneficial bacteria, such as those of the genus Bifidobacterium, can contribute to the activation of the defences against infections. On the other hand, the induction of the synthesis of high concentrations of regulating and anti-inflammatory cytokines can help for the development of a correct equilibrium in the responses of the lymphocytes Th1/h2, avoiding the pathological processes of chronic inflammation (Sanz et al. 2007 Probiotics as drugs against human gastrointestinal infections. Recent patents Anti-Infect Drug Disc. 2; 2:148-56). The abovementioned properties add additional value to the strains of Bifidobacterium in view of their application for improving the natural defences of the host and reduce the risk for disease, as it is proposed in the present invention.

### DESCRIPTION OF THE INVENTION

### Brief Description

The present invention contributes a new strain of the genus Bifidobacterium (IATA-ES2), the compounds freed for the supernatants of the cultivation and their formulation in the form of various preparations (functional and new foods, probiotics, symbiotics, supplements, nutraceutics and pharmaceutical formulations) with the capacity to modulate the composition of the glycoproteins of the epithelial intestinal cells which constitute the mucus and represent the first line of defence against harmful agents. The products object of the invention favour, in this way, the pattern of the glycosylation that is appropriate for a healthy intestine and for the residence of the beneficial intestinal microbiotics at the expense of the harmful one, thus reducing globally the risk of disease.

The bifidobacteria object of the invention, Bifidobacterium IATA-ES2, was isolated from the stool of healthy breastfed infants and identified by sequencing of the gene of the ARNr 16S and the gene tuf with the genus Bifidobacterium bifidum and differentiated by means of RAPDs of other strains belonging to the same species, thus demonstrating their exclusivity. The new strain secretes compounds capable of modulating the glycosylation pattern of the intestinal mucus, which constitutes the innate first line of defence, thus favouring the glycosylation pattern that is typical for a healthy intestine, characterised by elevated abundance of residues of galactosyl β(1→3) N-acetyl galactosamine (Gal β[1→3] GalNAc), demonstrated by the high level of staining with lectin PNA which recognises these residues (example 1, FIG. 1). The increase in the synthesis of these residues resulting from the activity of the bifidobacteria or its metabolites or other bacteria has not been demonstrated in any of the preceding studies (Freitas et al., 2001. A heat labile soluble factor from Bacteroides thetaiotaomicron VPI-5482 specifically increases the galactosylation pattern of HT29-MTX cells. Cell Microbiol. 3:289-300; Sanz et al. 2005. Indigenous microbes and their soluble factors differentially modulate intestinal glycosylation steps in vivo. Histochem. Cell. Biol. 124: 423-433). On the other hand, this strain does not favour especially the presence of residues, such as the fucose α (1,2) Gal β (1,4) GlcNAc, whose abundance is typical for patients with chronic inflammatory diseases (such as the celiac disease) and which favour the adhesion of pathogens, such as Helicobacter pylori, and are characterised by tinting with lectin UEA-I.

The modification of the glycosylation pattern produced by the strain and the products secreted in the medium acts also by favouring the adhesion of the species of the beneficial bifidobacteria, which are typical of breastfed infants and are used as probiotics, such as B. longum bv longum, B. longum bv infantis, B breve, B. bifidum and B. animalis, and reducing the adhesion of the pathogenic and pro-inflammatory bacteria such as Shigella (example 2; FIG. 2). The most significant increase in the adhesion of the epithelial cells is that of the strain itself that is object of the patent, demonstrating in this way the existence of a relationship of symbiosis with the host, which would favour its own colonisation. The compounds with biological activity secreted by the bifidobacterium object of the invention are of proteic nature and their molecular mass determined by means of ultrafiltration is greater than 30 kDa. Their biochemical characteristics do not coincide with those described previously in the case of the products secreted by Bacteroides thetaiotaomicron VPI-5482 (Freitas et al., 2005. Indigenous microbes and their soluble factors differentially modulate intestinal glycosylation steps in vivo. Histochem. Cell. Biol. 124: 423-433).

The bifidobacterium object of the invention (alive and inactivated) and its supernatants possess immunoregulatory and anti-inflammatory properties characterized by their stimulation of the synthesis of high concentrations of IL-10 and moderate concentrations of pro-inflammatory cytokines IL8, TNF-alpha and IFN-γ in peripheral blood mononuclear cells (PBMCs) and epithelial HT29-MTX blood cells. Furthermore, this strain reduces slightly the expression of the activation markers of the surface of the PBMCs, such as the co-receptors CD4 and CD8 and the co-stimulating molecule CD86, avoiding the excessive activation of the population of the lymphocytes T. The strain B. bifidum ES-2 induces, globally, the synthesis of mediators of the inflammation in balanced proportions which would permit it to activate the defences in a controlled form avoiding the development of excessive and chronic inflammation processes.

The strain object of the invention and the secreted products tolerate the gastrointestinal conditions and the technological processes thus guaranteeing their commercial exploitation. Their formulation includes, without limiting the scope of the invention, their incorporation in functional foods, new foods, supplements, nutraceutics, prebiotics, symbiotics and pharmaceutical formulations in individual or combined form with other microorganisms or functional ingredients of another nature.

Globally, the invention constitutes a natural method for improving the natural defences of the individual and reducing the risk for diseases by means of diverse applications targeted both to the general population and to groups at a risk to suffer from specific diseases such as infections or chronic inflammatory diseases. It is especially indicated for groups of the population with low defences, such as children and old-age people or persons with specific needs, since it can favour the colonisation of bifidobacteria typical for the newborns which increases our defences and regulates our immunological system. It also has the advantage that it does not have side effects unlike the pharmacological strategies (antibiotics, anti-inflammatory drugs, immunosupressors, etc.).

### DETAILED DESCRIPTION

The object of the present invention is a microorganism that is useful for the production of formulations, which improve the defences of the individual and reduce the risk of suffering from diseases, preferably microbial infections and immunological diseases, thanks to its ability to modulate the composition of the mucus and its interactions with the intestinal microbiotics, characterized in that it is not a genetically modified microorganism and is isolated and selected from the natural intestinal flora of healthy individuals. Hereinafter it shall be referred to as the microorganism object of the present invention.

Globally, the invention comprises a microorganism and its use for improving the natural defences of the individual and reducing the risk for diseases by means of diverse applications targeted both to the general population and to groups at a risk of suffering from specific diseases such as infections or chronic inflammatory diseases. It is especially indicated for groups of the population with low defences, such as children and old-age people or persons with specific needs, since it can favour the colonisation of bifidobacteria typical for the newborns which increases our defences and regulates our immunological system. It also has the advantage that it does not have side effects unlike the pharmacological strategies (antibiotics, anti-inflammatory drugs, immunosupressors, etc.).

The microorganism object of the present invention essentially comprises one strain of the genus Bifidobacterium. The advantages of the bifidobacterium, which is specifically selected for the formulation of pharmaceutical, probiotic, symbiotic, nutraceutic preparations or functional foods for increasing the defences of the individual, are multiple. The bifidobacteria have a special ability to colonise the intestinal tract of the newborns, contributing in this way significantly to the development of their defence mechanisms (immunological and of other nature) and the oral tolerance towards the antigens of the diet and the commensal microorganisms. This bacterial group is one of the main constituents of the intestinal microbiotics in the first years of life favoured by the maternal breastfeeding (representing up to 91%) and its predominance is associated with greater resistance to infections and lower susceptibility to immunological diseases (Harmsen, et al. 2000. Analysis of intestinal flora development in breast-feed and formula-feed infants by using molecular identification and detection methods. J. Pediatr. Gastroenterol. Nutr. 30: 61-67; Leteurtre et al. 2001. The family Bifidobacteriaceae. In: Dworkin, et al. (eds.), The Prokaryotes. pp. 1-70. Springer, N.Y.). At the same time, the strains of the genus Bifidobacterium are considered globally inductors of regulatory immunological responses with a less pro-inflammatory character than the lactobacilli and the other commensal bacteria (Zeuthen et al. 2006. Lactic acid bacteria inducing a weak interleukin-12 and tumour necrosis factor alpha response in human dendritic cells inhibit strongly stimulating lactic acid bacteria but act synergistically with gram-negative bacteria. Clin Vaccine Immunol. 13(3):365-75).

A specific object of the present invention consists of contributing a new strain of the genus Bifidobacterium bifidum and, in particular, Bifidobacterium IATA-ES2. One culture of Bifidobacterium IATA-ES2 has been deposited at the Spanish Type Culture Collection (CECT) with main office in Burjassot (Valencia) on Dec. 20, 2007, with a deposit number CECT 7365. This strain belongs to the genus B. bifidum according to the homology of the sequence of the gene of ARNr 16S and the gene tuf with others currently available in the databases (GenBank). It is an example of a strain of this species which possesses the properties and the applications object of the invention.

As an example, and without limiting the scope of the invention, the strain has been isolated proceeding from stools of healthy newborns and identified by sequentiation of the gene of ARNr 16S. The sequentiated fragment (1304 bases) was amplified by means of PCR using the primers Y1f and 1401r and, in addition, the primer 530f was used in the sequentiation in accordance with the procedures described by other authors (Satokari et al., 2001. Bifidobacterial Diversity in Human Feces Detected by Genus-Specific PCR and Denaturating Gradient Gel Electrophoresis. Appl. Environ. Microbiol. 67, 504-513; Favier et al. 2002. Molecular Monitoring of Succession of Bacterial Communities in Human Newborns. Appl. Environ. Microbiol. 68, 219-226). By comparing the obtained sequence with those existing in the databases, a maximum similarity was established with the equivalent sequences of the strains of the genus B. bifidum. A maximal identity (99%) was obtained with the strain Bifidobacterium bifidum KCTC 3202 deposited at the GenBank (access number U25952.1). At the same time, the identity was confirmed by sequentiation of the gene tuf. The sequentiated fragment was amplified by means of PCR by using the primers Btuf-1 and Btuf-2 in accordance with the procedure described by other authors (Ventura et al., 2003. Analysis, characterization, and loci of the tuf genes in lactobacillus and Bifidobacterium species and their direct application for species identification. Appl Environ Microbiol. 2003 November; 69(11):6908-22). Just as in the previous case, by comparing the obtained sequence (392 pb) with those existing in the databases, a maximal similarity (100%) was established with the equivalent sequence of the strain B. bifidum ATCC 29521 deposited at the GenBank (access number AY372041.1).

The differentiation of this strain from other strains of the same genus was made by studying the profile of the DNA obtained by means of PCR by making random amplifications with non-specific primers (RAPDS). For example, the use of the RAPD technique with the primer M13 has permitted to differentiate the strain ES-2 from the other two strains of the same genus isolated in the laboratory and from the collection strain B. longum LMG 1041T in at least 3 DNA strips. This indicates that the contribution is an exclusive strain.

The new strain object of this invention synthesizes and secretes in the culture medium compounds, which modulate the composition in carbohydrates of the glycoconjugates that form the intestinal mucus, and this leads to modifications in the interactions with the intestinal microbiotics.

The new strain object of the invention secretes compounds capable of modulating the glycosylation pattern of the intestinal mucus, which constitutes the first innate line of defence, thus favouring the glycosylation pattern that is typical for a healthy intestine characterised by elevated abundance of residues of galactosyl β(1→3) N-acetyl galactosamine demonstrated by the high level of staining with lectin PNA that recognises these residues (example 1, FIG. 1). For example, in the cells HT29-MTX, which constitute a model of intestinal cells, the addition of a free supernatant of cells of a culture of Bifidobacterium IATA-ES2 leads to tinting with lectin PNA up to 7 times greater than the one detected in the control (MRS) and with the supernatants of a culture of other species of bifidobacteria, such as B. animalis, B. longum and B. breve. The increase in the synthesis of these residues, to which lectin PNA is added by the activity of other bacteria or their metabolites, has not been demonstrated in any of the preceding studies (Freitas et al., 2001. A heat labile soluble factor from Bacteroides thetaiotaomicron VPI-5482 specifically increases the galactosylation pattern of HT29-MTX cells. Cell Microbiol. 3:289-300; Sanz et al. 2005. Indigenous microbes and their soluble factors differentially modulate intestinal glycosylation steps in vivo. Histochem. Cell. Biol. 124: 423-433). On the other hand, this strain does not favour especially the synthesis of residues typical for patients with chronic inflammatory diseases (for example, the celiac disease), such as Fucose α (1,2) Gal β (1,4) GlcNAc, which are characterized by a high level of tinting with lectin UEA-I, and it also favours the adhesion of pathogens such as Helicobacter pylori (example 1).

The modification of the glycosylation pattern produced by the strain and the products secreted in the medium acts also by favouring the adhesion of the species of the beneficial bifidobacteria, which are typical of breastfed infants and probiotics, such as B. longum bv longum, B. longum bv infantis, B breve, B. bifidum and B. animalis, and reducing the adhesion of opportunistic pathogens or potentially pro-inflammatory bacteria such as Shigella (example 2; FIG. 2). The most significant increase in the adhesion of the epithelial cells is that of the strain itself that is object of the patent, demonstrating in this way the existence of a relationship of symbiosis with the host, which favours its own colonisation.

The new strain produces compounds that are able to modulate the composition of the mucus and its interactions with the intestinal microbiotics. The compounds with biological activity, which are secreted by the bifidobacterium in the medium of the culture, are of proteic nature since their activity is destructed by the incubation as proteases, such as the proteinase K, but not by incubation in the presence de amilases or lipases. Their molecular mass determined by means of filtration is greater than 30 kDa. Therefore, their biochemical characteristics do not coincide with those described previously in the case of the products secreted by Bacteroides thetaiotaomicron VPI-5482 (Freitas et al., 2005. Indigenous microbes and their soluble factors differentially modulate intestinal glycosylation steps in vivo. Histochem. Cell. Biol. 124: 423-433).

In addition, the bifidobacterium object of the invention (alive or inactivated) and the products secreted to the medium of the culture possess characteristic immunoregulatory and anti-inflammatory properties for stimulating the synthesis of high concentrations of IL-10, and a moderate one for pro-inflammatory cytokines such as the TNF-α and IFN-γ in peripheral blood mononuclear cells (PBMCs), and also a moderate one for the chemokine IL8 in cells HT29-MTX (example 3, Table 1). The stimulation of the synthesis of pro-inflammatory cytokines (for example, TNF-α, IFN-γ) and of chemokine IL-8, which attracts phagocyte cells in order to activate the defences against the pathogen, is inferior compared to those generated by gram-negative bacteria such as Bacteroides and other evaluated bifidobacteria, and on the other hand, the synthesis of the anti-inflammatory cytokine IL-10, which would prevent the excessive activation of the mediators of the inflammation, is greater than the one that was detected in the majority of the strains. Furthermore, this strain reduces the expression of the markers for activation of the surface of the PBMCs, such as, for example, the co-receptors CD4 and CD8 and the molecule co-stimulator CD86, preventing the excessive activation of the population of cytotoxic lymphocytes T and T collaborators. In this way, it is demonstrated that the strain B. bifidum ES-2, induces, globally, the synthesis of mediators of the inflammation in balanced proportions which would permit it to activate the defences in a controlled form preventing the development of excessive and chronic inflammation processes.

The microorganism object of the invention belongs to one of the species of the genus Bifidobacterium identified predominantly in children, and to which beneficial properties are attributed for the health of the newborn as well as in administered probiotic form for other groups of the population in individual form or combined with other probiotics and prebiotics. For example, the administration of a given strain of the genus B. bifidum combined with other prebiotics was effective for the treatment of acute diarrhoea in children (Canani et al., 2007 Probiotics for treatment of acute diarrhoea in children: randomised clinical trial of five different preparations. BMJ. 2007 Aug. 18; 335(7615):340), a combination of Lactobacillus acidophilus and B. bifidum has demonstrated a significant effectiveness in the prevention of the traveller's diarrhoea (McFarland. 2007. Meta-analysis of probiotics for the prevention of traveler's diarrhea. Travel Med Infect Dis. Mar; 5(2):97-105), the administration of B. bifidum Bb12 to adults has increased the phagocytic capacity against pathogens (Schiffrin et al., 1997. Immune modulation of blood leukocytes in humans by lactic acid bacteria: criteria for strain selection. Am J Clin Nutr. 1997 August; 66(2):515S-520S). The consumption of Lactobacillus gasseri PA 16/8, B. longum SP 07/3, B. bifidum MF 20/5 during 3 weeks has reduced the episodes and the duration of the common cold (De Vrese, et al., 2005. Effect of Lactobacillus gasseri PA 16/8, Bifidobacterium longum SP 07/3, B. bifidum MF 20/5 on common cold episodes: a double blind, randomized, controlled trial. Clin Nutr. 2005 August; 24(4):481-91).

The strain object of the invention has a survival rate higher than 50% after its incubation in gastric conditions (saline solution with 3 g/l of pepsin, adjusted to pH 3.0), and a growth capacity of 70% in the presence of the concentrations of bile in the small intestine (0.5% oxgall, Sigma), which confirms that it is capable of maintaining its viability and metabolic activity in gastrointestinal conditions and, therefore, its possible probiotic use. It is also capable of growing in food samples, such as milk, which can act as a vehicle of the probiotics and serve as the origin of new functional foods. The strain object of the invention resists also the lyophilisation which ensures its commercialisation and possible administration in the form, among others, of pharmaceutical preparations.

The previously described active products released in the medium of the culture are stable with respect to the industrial and conservation treatments, such as, for example and without limiting the scope of the invention, congelation, concentration by filtration, chromatography, congelation and lyophilisation, which permits their formulation in the form of diverse preparations. The fact that the active compounds are secreted in the medium of the culture by the strain that is object of the invention ensures that its application does not require necessarily maintaining the viability of the bacterium in the product that is marketed which expands the number of the industrial applications.

The formulations prepared by using the microorganism object of the present invention can be developed industrially, which permits, among other things and without limiting the scope of the invention, different forms of presentation to the consumer: foods, new nutritional products, supplements, nutraceutics, pharmaceutical preparations, probiotics and/or symbiotics.

One particular object of the present invention comprises the use of the microorganism of the present invention or the bioactive compounds obtained in the process of preparation of the formulations in the form of food. In this way, the microorganism object of the present invention would form part of a food that is also prepared for providing, in addition to its normal nutritional value, a beneficial effect for reducing the risks for suffering from diseases.

Another particular object of the present invention comprises the use of the microorganism object of the same or the bioactive compounds derived in the process of making preparations in the form of nutraceutics defined as natural bioactive substances presented in a non-nutritious form.

Another object of the present invention is the use of the microorganism object of the same or the bioactive compounds derived for the purpose of obtaining a diet or nutritional supplement which would include in its composition the microorganism or the bioactive compounds derived from it in order to complement the diet with the purpose of healthy nutrition.

Another object of the present invention comprises the use of the microorganism or the obtained active compounds in the process of making pharmaceutical preparations. In this way, they would be used in the preparation of biologically active compositions which can be used as medicaments in order to achieve the described beneficial effects on the health.

Another additional object of the present invention is the use of the microorganism in the preparation of probiotics and/or symbiotics (combinations of probiotics and prebiotics) which would contain these microorganisms alive or lyophilised, would maintain their biological activity in the intestine, and would provide their beneficial effects if consumed in adequate amounts.

Another particular object of the present invention is its use as a new food, considering as such any food or ingredient which has not been used in a habitual form for consumption by people in the European Union since May 15, 1997.

One last object of the invention is its use in the form of foods, new foods, supplements, nutraceutics, pharmaceutical compositions, probiotics and/or symbiotics in combination with other microorganisms (probiotics or starter cultures) or other functional ingredients different from the live microorganisms.

### DESCRIPTIONS OF THE DRAWINGS

FIG. 1. Effect of the incubation of epithelial cells HT29-MTX, with compounds secreted to the supernatant of cultures of different bacteria, on the marking with lectin PNA marked with fluorescein which recognises the residues galactosyl β(1→3) N-acetyl galactosamine typical for the healthy intestine. Control (PBS); BH (control of bacteroides); MRS (control of bifidobacteria); Bac (Bacteroides spp.), ES1 (Bifidobacterium longum IATA-ES1); B. breve LMG11042T; ES2 (Bifidobacterium bifidum IATA-ES2), 324 (Bifidobacterium longum BIR-324), A2 (Bifidobacterium animalis). *The effect of the supernatant of ES2 was significantly different than that of the others in the case of applying the Mann-Whitney U test at P<0.05.

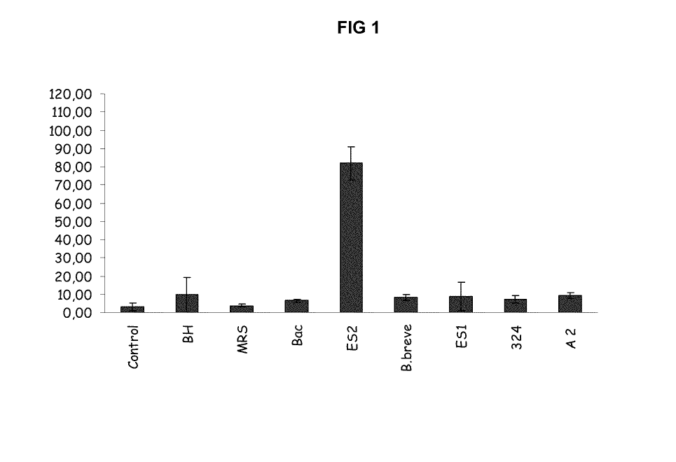

FIG. 2. Significant differences between the percentage of adhesion of B. bifidum ES2 and other intestinal bacteria to cells HT29-MTX after its stimulation with the compounds secreted to the supernatant of the culture by the same starting strain and with a sterile medium of the culture as a control (MRS) in the case of applying the Mann-Whitney test (*P<0.05).).

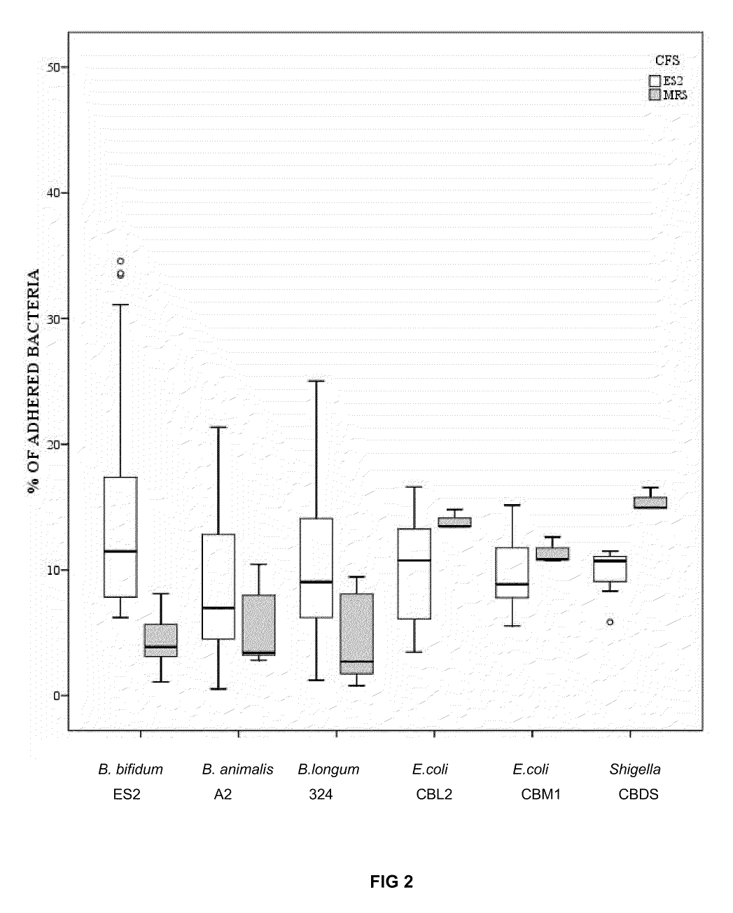

### EXAMPLES OF EMBODIMENT OF THE INVENTION

### Example 1

### Procedure of Evaluation of the Modulation of the Intestinal Glycosylation Pattern by Bacteria

### 1. Preparation of the Cultures and the Supernatants of Bifidobacteria and Others Intestinal Lactic Bacteria.

The strains of the genus Bifidobacterium were inoculated in 10 ml of breeding broth MRS (Scharlau Chemie S.A., Barcelona, Spain) containing 0.05% of flush (MRS-C) at 1% with cultivation for 24 h and were incubated for 22 h at 37° C. in anaerobiosis. (AnaeroGen; Oxoid, Basingstoke, UK). The strains of the genus Bacteroides were inoculated in breeding broth Brain Herat (Scharlau Chemie S.A., Barcelona, Spain) by following the same procedure. The cells were extracted by centrifugation (6.000 g, 15 min.), were flushed twice in PBS (10 mM sodium phosphate, 130 mM sodium chloride, pH 7.4), and were re-suspended in PBS containing 20% glycerol. Aliquots of these suspensions were frozen with liquid nitrogen and were conserved at −80° C. The number of viables after the cycle of freezing-unfreezing was determined by means of recounting in plates of MRSC and Schadler after incubation for 48 h. The viability was more than 90% in all cases. The supernatants of the medium of the culture were esterised by filtration (0.22-μm pore size, Millipore, Bedford, Mass.) in order to eliminate the possible presence of viable cells, and aliquots of these supernatants free of cells were conserved at −80° C. until their use.

### 2. Evaluation of the Glycosylation Pattern

The glycosylation pattern was determined by using the model of epithelial intestinal cells secretors of mucus HT29-MTX provided by Dr. Lesuffleur (INSERM U843, Paris, France). The cells were cultivated in the medium DMEM glutamax, containing 4.5 g/L D-glucose, 25 mM pyruvate (Gibco, Barcelona, Spain), 100 U/ml penicillin, 100 μl/ml streptomycin (Sigma, St. Louis, Mo.) and 10% bovine foetal serum (Gibco, Barcelona, Spain) inactivated for 30 minutes at 56° C. The cells were bred in jars of 75 cm² and plates with 6 and 24 bowls (Corning, Madrid, Spain) at 37° C. in atmosphere of 5% CO₂. The cultures were inoculated in the plates with a density of 5×10⁵ cells per ml. To detect the glycosylation pattern by marking with lectins, the cells HT29-MTX were incubated for 7 days in plates with 6 bowls until confluence was achieved. The monolayers were flushed with sterile PBS free of Ca²⁺ and Mg²⁺ (Gibco, Barcelona, Spain) and were incubated in the presence of fresh medium DMEM containing 20% (v/v) of the supernatant free of cells of the cultures of the cultivated bacteria or fresh medium BHI or MRS, used as controls, in the course of 7 additional days. The cells were incubated also in the presence of suspensions of bacterial cells added at a concentration of 10⁶ cfu/ml for 2 days. After the evaluation of the cells HT29-MTX, they were flushed 3 times with PBS free of Ca²⁺ and Mg²⁺ PBS (Gibco, Barcelona, Spain) and were incubated for 5 minutes at 37° C. with 0.25% trypsin and 1 mM EDTA, re-suspended in DMEM and, afterwards, they were centrifuged to extract the cells (1800 r.p.m., 10 min., at 4° C.). The cells were flushed twice with PBS and were tinted with lectins marked with fluorescein (FITC) for 40 minutes. The lectins that were used and their concentrations were the following: UEA (Ulex europaeus) [α(1,2)-fucose] (Sigma), 50 μg/ml; PNA (Arachys hypogaea, β-gal(1→3)galNAc (Sigma), 10 μg/ml; HPA (Helix pomatia), α-NAc residues (Molecular Probes, Barcelona, Spain), 10 μg/ml; SBA (Gycine max), α- y β-Nac. a galactopyranosyl (Molecular Probes, Barcelona, Spain), 10 μg/ml. Afterwards the cells were flushed 3 times and were re-suspended in 400 μl PBS. The grade of tinting with the lectins was quantified in a flow cytometer (EPICS® XL-MCL flow cytometer; Beckman Coulter, Fla., US).

### Example 2

### Procedure of Evaluation of the Adhesion of Bacteria by Using a Model of Epithelial Intestinal Cells

The suspensions of bacteria were tinted by incubation with 10 mM 5-diacetate of carboxyfluorescein (Sigma, St. Louis, Mo.), at 37° C., during 1 hour. The suspensions of marked cells were adjusted to optical density of 0.50±0.05 at 600 nm and were added to confluent cultures of cells HT29-MTX and were then incubated at 37° C. during 1 hour. The cells were flushed with PBS to eliminate the bacteria that have not adhered, and the adhered ones were extracted by means of incubation with 200 μl of 1% SDS (Sigma, St Louis, Mo.) in 0.1 M NaOH at 37° C. during 1 hour. The released fluorescence was measured in a fluorimeter of multi-bowl plates (Fluoroskan Ascent, Labsystem, Oy, Finland) at 485 nm and 538 nm of excitation and emission wavelengths, respectively. The adhesion was expressed as a percentage of the fluorescence obtained after the adhesion of the bacteria to the cells HT29-MTX with respect to the initial fluorescence of the bacterial suspension added per bowl.

### Example 3

### Procedure of Evaluation of the Immunological Properties of Bifidobacterium bifidum ES-2

### 1. Preparation of the Cultures and the Supernatants of Bifidobacteria and Other Intestinal Bacteria.

The strains were inoculated in 10 ml of breeding broth MRS (Scharlau Chemie S.A., Barcelona, Spain) containing 0.05% of flush (MRS-C) at 1% with cultivation for 24 h and were incubated for 22 h at 37° C. in anaerobiosis (AnaeroGen; Oxoid, Basingstoke, UK). The cells were extracted by centrifugation (6.000 g, 15 min.), were flushed twice in PBS (10 mM sodium phosphate, 130 mM sodium chloride, pH 7.4), and were re-suspended in PBS containing 20% glycerol. Aliquots of these suspensions were frozen with liquid nitrogen and were conserved at −80° C. The number of viables after the cycle of freezing-unfreezing was determined by means of recounting in plates of MRSC and Schadler after incubation for 48 h. The viability was more than 90% in all cases. Each aliquot was used for one test only. With the purpose of evaluating the effects of dead bacteria, some of the aliquots were inactivated by freezing (3 cycles of congelation at −20° C. and decongelation) and per heat (30 min at 80° C.). The pH values of the obtained supernatants were adjusted to 7.2 with NaOH and they were esterised by filtration (0.22-μm pore size, Millipore, Bedford, Mass.) in order to eliminate the possible presence of viable cells. The aliquots of the supernatants free of cells were conserved at −80° C. until their use.

### 2 Isolation and Stimulation of PBMCs.

The PBMCs were isolated from the peripheral blood of 4 healthy volunteers (average age of 30 years, interval 24-40) in tubes with heparin. The isolation of the PBMCs was performed by centrifugation in a gradient of Ficoll (Amersham Biosciences, Piscataway, N.J.). The cells were flushed with medium RPMI 1640 (Cambrex, N.Y., USA) and were adjusted to a density of 1×10⁶ cells/ml in medium RPMI 1640 containing, in addition, 10% bovine foetal serum (Gibco, Barcelona, Spain), 2 mM L-glutamine, 100 μg/ml streptomycin and 100 U/ml penicillin (Sigma). The PBMCs were incubated in flat-bottom polystyrene plates with 24 bowls (Corning, Madrid, Spain) in the presence or the absence of stimulating agents at 37° C., with 5% CO₂, during 24 h. Alive and dead bacterial suspensions of 1×10⁶ CFU/ml and volumes of supernatants of 150 μl were used as a stimulus. Purified lipopolysaccharide (LPS) of E. coli O111:B4 (Sigma, St. Louis, Mo.) with concentration of 1 μg/ml was used as a positive control. The production of cytokines in non-stimulated PBMCs was used as a negative control. Each type of stimulus was tested twice in each experiment. The supernatants of the cultures were collected by centrifugation, were fractioned and stored in aliquots at −20° C. until the detection of cytokines.

### 3. Stimulation of HT29-MTX Cells.

The intestinal epithelial cells secretors of mucus HT29-MTX were cultivated, as described in example 1, and were subsequently incubated in the presence of cellular suspensions and cultivated supernatants free of cells of bifidobacteria and other intestinal bacteria during 24 h by following the same process described for the stimulation of PBMCs. After this period, the supernatants of the cultures were collected by centrifugation, were fractioned and stored in aliquots at −20° C. until the detection of cytokines.

### 4. Determination of the Cytokines.

The concentrations of cytokines (IL-8, IL-1, IFN-γ, IL-10 and TGF-β) of the supernatants were measured with ELISA kits of Bioscience (BD Biosciences, San Diego, Calif.) according to the instructions of the trading house.

TABLE 1
Table 1. Immunomodulating properties of the bifidobacteria and other intestinal
lactic bacteria. Effect of the viable cells on the production of cytokins
(pg/ml) by PBMCs and viable cells and cultivated supernatants on the production
of chemiokine IL8 by epithelial cells HT29MTX.

| | Cytokines (pg/ml) | | | | |
|---|---|---|---|---|---|
| | IL-1 | INF-γ | IL-10 | IL-8 | IL8 |
| Stimulus | Cells | Cells | Cells | Cells | Remaining |
| RPMI | ND | 9.0 (1.0) | 58.0 (3.0) | 488.5 (5.5) | 488.5 (5.5) |
| LPS | ND | 12.0 (0.5) | 399.0 (8.0) | — | — |
| Bacteroides | — | — | — | 937.9 (80.9) | 2500.2 (52.1) |
| ¹A2 | 255.0 ± 1.0 | 13.0 ± 2.0 | 699.0 ± 396.0 | 461.1 (48.2) | 462.0 (50.8) |
| ²ES2 | 65.0 ± 4.0 | 52.0 ± 12.0 | 1091.0 ± 329.0 | 837.0 (60.4) | 639.7 (21.8) |
| ³BIR-324 | ND | 11.0 ± 5.0 | 469.0 ± 15.0 | 1558.8 (21.9) | 935.2 (83.6) |
| ⁴W11 | — | 160.4 ± 6.8 | 486.0 ± 236.4 | — | |
| ⁵BB536 | — | 143.7 ± 18.3 | 1390.0 ± 268.8 | — | |

ND, not detected
—, not evaluated
¹Bifidobacterium animalis IATA-A2,
²Bifidobacterium bifidum IATA-ES2,
³Bifidobacterium longum BIR-324,
⁴Bifidobacterum longum W11,
⁶Bifidobacterum longum BB536.

1-21. (canceled)

22. A microorganism of Bifidobacterium bifidum species with deposit number CECT 7365.

23. The microorganism according to claim 22, wherein the microorganism is in the form of viable cells.

24. The microorganism according to claim 22, wherein the microorganism is in the form of non-viable cells.

25. A combination of microorganisms comprising the microorganism according to claim 22 and one or more other microorganisms.

26. Secreted compounds and/or metabolites obtained from the microorganism according to claim 22.

27. A food comprising the microorganism according to claim 22.

28. A food comprising the secreted compounds and/or metabolites according to claim 26.

29. A food comprising the microorganism according to claim 22, wherein the food is a probiotic product, symbiotic product, supplement or nutraceutical.

30. A food comprising the secreted compounds and/or metabolites according to claim 26, wherein the food is a probiotic product, symbiotic product, supplement or nutraceutical.

31. A pharmaceutical composition comprising the microorganism according to claim 22.

32. A pharmaceutical composition comprising the secreted compounds and/or metabolites according to claim 26.

33. A method of using the microorganism according to claim 22 for the prevention and/or treatment of at least one microbial infection.

34. A method of using the microorganism according to claim 22 for the prevention and/or treatment of immunological diseases.

35. A method of using the microorganism according to claim 22 for the production of compounds with capacity to modulate the composition of the mucus.

36. A method of using the microorganism according to claim 22 for the prevention and/or treatment of celiac disease.

37. A method of using the microorganism according to claim 22 for increasing the adhesion to epithelial cells of bacterial species beneficial to the health.

38. A method of using the microorganism according to claim 22 for reducing the adhesion to epithelial cells of pathogenic bacteria.

39. A method of using the microorganism according to claim 22 for stimulating the synthesis of cytokines and reducing the cellular markers of activation.

40. A method of using the microorganism according to claim 22 for stimulating the synthesis of anti-inflammatory cytokine IL-10, and the proinflammatory cytokines and chemokines IL8, TNF-alpha and IFN-γ; and reducing the cellular markers of activation CD4, CD8, and CD86.

41. A method of using the microorganism according to claim 22 for the preparation of a functional food, a probiotic product, a symbiotic product, a supplement, a nutraceutical, or a pharmaceutical composition.


SEQUENCE LISTING (2 sequences)

SEQ ID NO: 1 - DNA, 1304 bp, Bifidobacterium bifidum
ccatcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagatttcatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccracgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggtctgta

SEQ ID NO: 2 - DNA, 392 bp, Bifidobacterium bifidum
cgcagcgtggtatcaccatcaacatcgcccacatcgagtaccagaccgcgaagcgtcactacgctcacgtggactgcccgggccacgccgacttcgtgaagaacatgatcaccggtgctgcccagatggatggcgccatcctcgttgtggccgccaccgacggcccgatggcccagacccgcgagcacgttctgctcgcccgtcaggtgggtgtcccgcgtatcctcgtcgccctgaacaagtgcgacatggtcgaggacgaggagctcatcgagctcgttgaggaagaggtccgtgacctcctcgacgagaacggcttcgaccgcgactgcccggtcatccgtacctccgcctacggcgctctgcacgatgacgctccggaccacgacaag